(19) United States
(12) Patent Application Publication
(10) Pub. No.: US 2024/0261291 A1
(43) Pub. Date: Aug. 8, 2024

(54) Ophthalmic Compositions and/or Methods for Presbyopia, Mydriasis and/or Ocular Discomfort Management

(71) Applicant: Brien Holden Vision Institute Limited, Sydney, NSW (AU)

(72) Inventor: Padmaja Rajagopal Sankaridurg, Maroubra, NSW (AU)

(21) Appl. No.: 18/567,653

(22) PCT Filed: Jun. 10, 2022
(86) PCT No.: PCT/IB2022/055399

§ 371 Date: Dec. 6, 2023

Related U.S. Application Data
(60) Provisional application No. 63/209,627, filed on Jun. 11, 2021.

(51) Int. Cl. A61K 31/522; A61K 9/00; A61K 45/06; A61P 27/10

(52) U.S. Cl. CPC A61K 31/522; A61K 9/0048; A61K 45/06; A61P 27/10

(57) ABSTRACT

An ophthalmic composition comprising: an adenosine receptor antagonist (e.g., caffeine or a related compound) or a combination of an adenosine receptor antagonist (e.g., caffeine or a related compound) and one or more parasympathomimetics and/or muscarinic receptor agonists, and methods of using the same.

### CROSS REFERENCE

This application claims the benefit of U.S. Provisional Application No. 63/209,627, filed Jun. 11, 2021, the disclosure of which is incorporated by reference herein in its entirety.

### TECHNICAL FIELD

This disclosure relates to ophthalmic compositions and/or methods for the management of presbyopia and/or mydriasis. The ophthalmic compositions may comprise an adenosine receptor antagonist, e.g., caffeine or a related compound. The compositions may comprise a combination of an adenosine receptor antagonist, e.g., caffeine or a related compound, and one or more parasympathomimetics and/or muscarinic receptor agonists. The ophthalmic compositions and methods provided herein may be directed for delivery to the eye and/or surrounding adnexa for improving vision conditions caused by presbyopia and/or mydriasis and/or ocular disorders requiring modulation of light received by the eye.

### BACKGROUND

Presbyopia is a condition where there is an age-related decline in the ability of the eye to focus on objects at near distances. The condition is usually apparent between ages 40 to 45 and progressively declines with age, requiring the use of optical aids and in some instances, chemical compositions and surgical methods to restore near vision. The mechanisms underlying presbyopia are not well understood and have been attributed to change in refractive indices, modulus of elasticity, hardening of crystalline lens of the eye as well as changes in the function of the ciliary body. These changes result in a decrease in the accommodative ability, i.e., a change in the curvature of the anterior surface of the crystalline lens when focusing from distant to near objects and therefore result in increasingly blurred vision for near distances. A decrease in the pupillary diameter of the eye may increase the depth of focus and restore near vision and indeed there have been several techniques that have tried to mimic a reduced pupil size such as, use of pinhole spectacles, corneal inlays, and use of pharmaceutical agents such as pilocarpine and aceclidine. Such agents are known to trigger ciliary spasm and induce headaches or similar undesirable conditions.

Mydriasis is a state of the eye characterized by a larger than usual pupillary diameter that is mostly unresponsive or sluggishly responsive to light and commonly results from administration of a pharmaceutical agent and in certain other situations, may accompany an ocular disorder. Most commonly, it occurs because of one or more of mydriatic or cycloplegic agents that are used to increase the pupillary size to facilitate ocular examination. Depending on the type, dose and frequency of the agent used, the increased pupillary diameter may persist for few hours to days post instillation and may be associated with disabling symptoms such as photophobia and or an inability to focus at near objects. Thus, there is a need to shorten the duration over which the pupil remains dilated and/or reduce the effect on the pupillary diameter/mydriasis to reduce the side effects of mydriasis.

Individuals with excessive ocular aberrations, distorted, scarred or post-surgical corneas, or eyes with post refractive surgery experience light and vision disturbances including glare, reduction in low contrast vision, ocular discomfort and/or night vision disturbances including haloes and starbursts around lights. In these eyes, there is a need to reduce the pupillary response to illumination to reduce the discomfort related to symptoms.

Accordingly, there is a need for an ophthalmic composition and/or ophthalmic devices containing or delivering the such pharmaceutical compositions, and/or methods of using the such pharmaceutical compositions, or combinations thereof, for effectively managing the visual deficits that accompany presbyopia without the risk of adverse side effects, managing the side effects of mydriasis of the pupil of the eye or minimizing the effect of vision disturbances and ocular discomfort in those with need.

### SUMMARY

Some embodiments described herein provide ophthalmic compositions, and/or methods of delivery and/or treatment, to prevent, control, slow, reduce, retard, and/or mitigate the vision related deficits that accompany presbyopia and/or mydriasis and/or eyes with disorders requiring modulation of light received at the eye. In some embodiments, the pharmaceutical composition provided herein is an ophthalmic composition.

Some embodiments described herein provide ophthalmic compositions, and/or methods of delivery and/or treatment, to improve vision in presbyopia and/or during use of mydriatic agents and/or in disorders of requiring modulation of light received at the eye.

In some embodiments, provided herein is an ophthalmic composition, comprising an adenosine receptor antagonist or a related compound.

In some embodiments, provided herein is an ophthalmic composition, comprising one or more of an adenosine receptor antagonist and one or more of a parasympathomimetic agent.

In some embodiments, provided herein is an ophthalmic composition comprising one or more of an adenosine receptor antagonist and an alpha 1-antagonist.

In some embodiments, the adenosine receptor antagonist, or the one or more of an adenosine receptor antagonist, is a xanthine compound.

In some embodiments, the xanthine compound is a methylxanthine compound.

In some embodiments, the xanthine compound is caffeine (1,7 methylxanthine), 7-methylxanthine; 1,7-dimethylxanthine (paraxanthine), 3,7-dimethylxanthine (theobromine); 7-methylxanthine (heteroxanthine), 3-methylxanthine; 1-methylxanthine, isobutylmethylxanthine (IBMX); 1-Hexyl-3,7-dimethylxanthine (pentifylline); and 1,7-dimethylxanthine.

In some embodiments, provided herein is an ophthalmic delivery system comprising an ophthalmic composition comprising an adenosine receptor antagonist for delivery to the eye and/or the surrounding adnexa of the eye.

In some embodiments, provided herein is an ophthalmic delivery system comprising an ophthalmic composition comprising one or more of an adenosine receptor antagonist and a parasympathomimetic agent for delivery to the eye and/or the surrounding adnexa of the eye.

In some embodiments, provided herein is a method of treating presbyopia and/or mydriasis and/or a disorder of the eye requiring modulation of light received at the eye in a subject, the method comprising administering an ophthalmic composition comprising an adenosine receptor antagonist to the eye and/or the surrounding adnexa of the eye of the subject.

In some embodiments, provided herein is a method of treating presbyopia and/or mydriasis and/or a disorder of the eye requiring modulation of light received at the eye in a subject, the method comprising administering an ophthalmic composition comprising one or more of an adenosine receptor antagonist and a parasympathomimetic agent to the eye and/or the surrounding adnexa of the eye of the subject.

In some embodiments, provided herein is a method of treating presbyopia and/or mydriasis and/or a disorder of the eye requiring modulation of light received at the eye in a subject, the method comprising administering an ophthalmic composition comprising a therapeutically effective amount of one or more of an adenosine receptor antagonist to the eye and/or the surrounding adnexa of the eye of the subject.

In some embodiments, provided herein is a method of treating presbyopia and/or mydriasis and/or a disorder of the eye requiring modulation of light received at the eye in a subject, the method comprising administering an ophthalmic composition comprising a therapeutically effective amount of one or more of an adenosine receptor antagonist and one or more of a parasympathomimetic agent to the eye and/or the surrounding adnexa of the eye of the subject.

In some embodiments of the ophthalmic composition, the ophthalmic device, or the method of treating, as disclosed herein, the adenosine receptor antagonist is a xanthine related compound or a combination of xanthine related compounds, wherein the xanthine related compound is selected from the group consisting of: caffeine (1,7 methylxanthine), 7-methylxanthine; 1,7-dimethylxanthine (paraxanthine), 3,7-dimethylxanthine (theobromine); 7-methylxanthine (heteroxanthine), 3-methylxanthine; 1-methylxanthine, isobutylmethylxanthine (IBMX): 1-Hexyl-3,7-dimethylxanthine (pentifylline); and 1,7-dimethylxanthine.

In some embodiments of the ophthalmic composition, the ophthalmic device, or the method of treating, as disclosed herein, the parasympathomimetic agent is neostigmine, cevimeline, pilocarpine, aceclidine, carbachol, methacholine, echothiophate, physostigmine, prostigmine, phospholine iodide, demecarium bromide, or related compounds. In some embodiments, the parasympathomimetic agent is a combination of one or more of neostigmine, cevimeline, pilocarpine, aceclidine, carbachol, methacholine, echothiophate, physostigmine, prostigmine, phospholine iodide, demecarium bromide, or related compounds.

In some embodiments, the ophthalmic composition comprises a therapeutically effective amount of an adenosine receptor antagonist, e.g., caffeine and/or related compound, and the concentration of the adenosine receptor antagonist, e.g., caffeine and/or related compound, is in the range of from about 0.5 w/v. % to about 10 w/v. % of the composition.

In some embodiments, the ophthalmic composition comprises a therapeutically effective amount of an adenosine receptor antagonist, e.g., caffeine and/or related compound, at about 0.5 w/v. % to about 10.0 w/v. % of the composition, and may optionally further comprise any combination of one of more of a parasympathomimetic agent, a muscarinic receptor agonist, an alpha-adrenergic antagonist, a vasoconstricting agent or an anti-inflammatory agent.

In some embodiments, the ophthalmic composition comprises a therapeutically effective amount of an adenosine receptor antagonist, e.g., caffeine or related compound, at about 0.5 w/v. % to about 10 w/v. % of the composition, and may optionally further comprise any combination of one of more of a parasympathomimetic agent, a muscarinic receptor agonist, an alpha-adrenergic antagonist, a vasoconstricting agent or an anti-inflammatory agent, wherein the composition has a pH in the range of about 4.0 to about 8.0, such as about 4.0 to about 7.5.

In some embodiments, the use of the ophthalmic composition as provided herein, reduces the effective pupillary diameter of the eye by approximately 0.1 mm to about 3.5 mm, such as by approximately 0.2 mm to about 3.5 mm. In some embodiments, the use of an ophthalmic composition comprising a therapeutically effective amount of an adenosine receptor antagonist, e.g., caffeine or a related compound, reduces the effective pupillary diameter of the eye by approximately 0.2 mm to about 3 mms.

In some aspects of the invention, provided herein is a method of treating presbyopia and/or mydriasis and/or a disorder of the eye requiring modulation of light received at the eye of a subject, the method comprising administering to the subject an ophthalmic composition comprising a therapeutically effective amount of an adenosine receptor antagonist, such as one or more adenosine receptor antagonists. In some embodiments, the adenosine receptor antagonist is a xanthine compound, for example, is a methylxanthine compound. In some embodiments, the ophthalmic composition comprises at least one xanthine compound. In some embodiments, the xanthine compound, for example, the methylxanthine compound, is selected from the group consisting of caffeine (1,7 methylxanthine), 7-methylxanthine; 1,7-dimethylxanthine (paraxanthine), 3,7-dimethylxanthine (theobromine); 7-methylxanthine (heteroxanthine), 3-methylxanthine; 1-methylxanthine, isobutylmethylxanthine (IBMX); 1-Hexyl-3,7-dimethylxanthine (pentifylline); and 1,7-dimethylxanthine; or combinations thereof.

In some aspects of the invention, the method of treating provided herein comprises administering the ophthalmic composition disclosed herein to the eye of the subject either simultaneously or sequentially with administration of a mydriatic or a cycloplegic agent to the eye of the subject.

In some aspects of the invention, provided herein is a method of managing or treating an eye of a subject following refractive surgery or a distorted, scarred or astigmatic cornea, the method comprising administering the ophthalmic composition disclosed herein to the eye of the subject, wherein the method reduces one or more side effects related to the disorder of the eye.

In some embodiments, the methods of treating provided herein comprises administering an ophthalmic composition comprising a therapeutically effective amount of an adenosine receptor antagonist, e.g., caffeine or a related compound, such as in an amount of about 0.5 w/v. % to about 10 w/v. % of the composition, to at least one eye of a subject to improve the performance of one or more visual performance measures in the at least one eye of the subject. In some embodiments, the method improves the performance of one or more visual performance measures in the at least one eye of said subject. In some embodiments, the one or more visual performance measures is selected from the group consisting of high or low contrast distance visual acuity, high or low contrast intermediate visual acuity, high or low contrast near visual acuity, haloes, and glare and night vision.

Other features and advantages of the subject matter described herein will be apparent from the description and drawings, and from the claims provided herein.

### BRIEF DESCRIPTION OF THE DRAWINGS

Aspects of the embodiments described herein may be best understood from the following detailed description when read with the accompanying figures.

FIG. 1 is a schematic of the pupil diameter of eyes prescribed with caffeine 2% over time.

FIG. 2 illustrates the increase in depth of field with reducing pupil diameter (adapted from Chap 19, Depth of Field from Optics of the Human eye, Eds Atchison D A and Smith G, 2000).

FIG. 3: Table illustrates the pH over an extended period of time for an aqueous ophthalmic composition comprising Caffeine.

FIG. 4 illustrates that difference in pupil diameter in the right and left eyes of an individual, wherein Tropicamide 1% was instilled in both eyes and approximately 50 minutes later, an ophthalmic composition comprising Caffeine at 2 w/v. % was instilled in the left eye only.

### DETAILED DESCRIPTION

The following disclosure provides many different embodiments, or examples, for implementing different features of the provided subject matter. Specific examples of components and arrangements are described below to simplify the present disclosure. These are, of course, merely examples and are not intended to be limiting. In addition, the present disclosure may repeat reference numerals and/or letters in the various examples. This repetition is for the purpose of simplicity and clarity and does not in itself dictate a relationship between the various embodiments and/or configurations discussed.

The subject headings used in the detailed description are included for the ease of [symbol] and should not be used to limit the subject matter found throughout the disclosure or the claims. The subject headings should not be used in construing the scope of the claims or the claim limitations.

The terms “about” as used in this disclosure is to be understood to be interchangeable with the term approximate or approximately.

The term “comprise” and its derivatives (e.g., comprises, comprising) as used in this disclosure is to be taken to be inclusive of features to which it refers, and is not meant to exclude the presence of additional features unless otherwise stated or implied.

Terms are used herein as generally used in the art, unless otherwise defined in the following:

The term “presbyopic eye” is understood to refer to an eye that is already presbyopic, is pre presbyopic, or is progressing towards presbyopia.

The term “mydriasis” is understood to refer to an eye that has a pupil diameter that is larger relative to the normal pupil diameter or relative to the baseline pupil diameter and resulting from either the use of an eye drop that enlarges the pupil or may be the result of a side effect of either a topical or systemic medication or resulting from other conditions such as an injury to the eye.

The term “disorder requiring pupil modulation” is understood to refer to an eye that has a disease or disorder or a condition wherein the eye may be experiencing one or more of vision disturbances such as reduced vision, glare, photophobia, haloes or night vision disturbances and may be the result of a procedure such as refractive surgery, condition such as corneal astigmatism, high ocular aberrations, scarring or disease states.

The terms “treating” (or “treat” or“treatment”), unless otherwise specified, includes the generally accepted meaning which encompasses preventing, controlling, slowing, reducing, retarding, and/or mitigating, a symptom associated with a condition (e.g., presbyopia and/or mydriasis and/or a disorder requiring pupil modulation) and/or progression of a condition (e.g., presbyopia, such as the progression of presbyopia in an eye of a patient). Treatment may include therapeutic and/or prophylactic administration (e.g., of an ophthalmic composition or an device impregnated with the pharmaceutical composition, as disclosed herein). For example, treatment of an eye that is already presbyopic (or at risk of developing presbyopia), in a patient diagnosed as having myopia (high, moderate, or low) or pre-presbyopic (at risk at developing presbyopia), or an eye that has undergone refractive surgery may include, but is not limited to, preventing, controlling, slowing, reducing, retarding, or mitigating, the vision related deficits that accompany the condition.

The term “ophthalmic device” is understood to refer to an object that is placed on or resides in or more layers or in the eye and adnexa. The device may provide optical correction. An ophthalmic device includes, but is not limited to, a contact lens(es), an ocular insert(s), a corneal onlay(s), a nano wafer(s), a liposome(s), a nanoparticle(s), a punctal plug(s), or a hydrogel matrix(ces) with microfluid reservoir.

The term “adenosine receptor antagonist” or “adenose receptor antagonist” or “adenosine receptor inhibitor” refers to chemical composition or agent(s) that act on or block the adenosine receptors or prevent or antagonize or reduce or slow the action of adenosine receptor agonists. At least four subtypes of adenosine receptors called A1, A2a, A2b, and A3, respectively are known. Adenosine receptors are members of the G-protein coupled receptor (GPCR) superfamily, considered to mediate stimulation or inhibition of adenylyl cyclase activity, and hence cyclic AMP levels include both naturally occurring peptides, as well as adenosine receptor fragments and variants that retain full or partial adenosine receptor biological activity.

The term “subject” to which administration is contemplated, can be an animal, including, but not limited to, a human (e.g., a male or female of any age group, such as an adult subject or an adolescent subject); primates (e.g., cynomolgus monkeys, rhesus monkeys), and/or other mammals, including commercially relevant mammals such as cattle, pigs, horses, sheep, goats, cats, dogs, rabbits, rodents, and/or birds. In some embodiments, the subject is a mammal. In some embodiments, the subject is a human. In some embodiments, the subject is an adolescent human. In some embodiments, the subject is a patient, for example, a human patient.

The term “therapeutically effective amount” means that when the compound or composition or chemical is administered to the individual in need for management and/or treatment, the amount of the compound or composition or chemical is substantially sufficient to produce the intended result for management and/or treatment of the condition or improve one or more of the visual performance measures.

The term “pharmaceutically acceptable form” refers to a pharmaceutically acceptable salt, solvate or prodrug thereof. The term“pharmaceutically acceptable salt” refers to those salts which are, within the scope of sound medical judgment, suitable for use in contact with the tissues of subjects without undue toxicity, irritation, allergic response, and the like, and are commensurate with a reasonable benefit/risk ratio. Pharmaceutically acceptable salts are well known in the art. For example, pharmaceutically acceptable salts are described in detail in Berge, S. M. et al. J. Pharmaceutical Sciences. 1977, 66(1):1-19. Pharmaceutically acceptable salts of the compounds provided herein include those derived from suitable inorganic and organic acids and bases, such as suitable inorganic and organic addition acids and bases. Pharmaceutically acceptable organic salts include salts prepared from organic acids such as acetic, lactic, malic, tartaric, citric, ascorbic, and the like and inorganic salts such as chloride, phosphates, sulfates and the like or salts from buffering agents as for example, phosphate buffering agents, citrate buffering agents, acetate buffering agents, carbonate buffering agents, organic buffering agents, acetate buffering agents, aminoacid buffering agents boric acid; sodium borate; potassium citrate; citric acid and the like.

Presbyopia or pre-presbyopia may be characterized by a decline in accommodation and thus the difficulty in focusing and looking at objects at near distances increases with age. In such instances, commonly the person resorts to the use of an optical device or magnification aids that compensate for the decline in accommodation. However, the condition is progressive and can significantly impact multiple domains of quality of life, such as for example performing day to day activities, cosmesis etc. The current treatments for presbyopia such as multifocal spectacles or contact lenses suffer from limitations such as reduced field of view with progressive addition spectacles or decreased quality of vision and symptoms of ghosting and haloes with multifocal lenses. There exists a need for better treatments for presbyopia either in isolation or in combination with other treatment modalities.

Mydriasis is an abnormal state of the eye characterized by a pupil that is larger relative to the normal pupil size of the individual. The condition is commonly a result of mydriatic or cycloplegic agents used as part of an ocular examination in a doctor's office or a result of a pharmacological agent, such as for example certain anti histamines or may be a result of a pathological condition of the eye. Due the larger than normal pupil diameter, greater amount of light is received at the photosensitive layer of the eye called the retina and may result in significant discomfort to the individual. During daylight or bright lights the condition may resulting in symptoms of glare, sensitivity to light, reduced low contrast visual acuity and inability to focus at intermediate to near distances as well as foggy vision. During low light or night, it may result in significant night vision disturbances such as haloes and starbursts around lights. These symptoms may persist for hours to days and therefore result in significant disability to the individual. Although there exist some pharmacological compositions that aid in reversal of mydriasis these may result in side effects such as spasms of the muscle of iris and ciliary body resulting in brow pain and headaches, ocular redness and may also result in a fixed, pinpoint pupil unresponsive to light. Therefore, there exists a need for improved treatments for mydriasis.

In certain ocular conditions of the eye such as aberrated or scarred corneas or highly astigmatic corneas as in keratoconus or corneal dystrophies or in eyes that have undergone refractive surgery such as LASIK or in pathological states of the eyes that result in large pupils, the individual may experience symptoms similar to those eyes with mydriasis. During daylight or bright lights the condition may result in symptoms of glare, sensitivity to light, reduced low contrast visual acuity and inability to focus at intermediate to near distances as well as foggy vision. During low light or night, it may result in significant night vision disturbances such as haloes and starbursts around lights. These symptoms may persist for hours to days and therefore result in significant disability to the individual. Therefore, there exists a need for improved treatments for these ocular conditions.

Some embodiments described herein relate to an ophthalmic composition comprising an adenosine receptor antagonist, such as a therapeutically effective amount of an adenosine receptor antagonist. Some embodiments described herein relate to methods of delivery and/or methods of treatment administering the ophthalmic composition provided herein to prevent, control, slow, reduce, retard, and/or mitigate the vision related deficits that accompany presbyopia and/or mydriasis and/or eyes with disorders requiring light modulation. In some embodiments, the adenosine receptor antagonist may be a non-selective adenosine antagonist. In some embodiments, the adenosine receptor antagonist may be a non-selective antagonist acting on one or more of adenosine receptors subtypes of A₁, A_{2A}, A_{2B} or A₃. In some embodiments, the adenosine receptor antagonist is a xanthine compound or a methylxanthine compound, or a pharmaceutically acceptable form thereof. In some embodiments, the adenosine receptor antagonist is a xanthine compound or a methylxanthine compound selected from the group consisting of: caffeine (1,7 methylxanthine), 7-methylxanthine; 1,7-dimethylxanthine (paraxanthine), 3,7-dimethylxanthine (theobromine); 7-methylxanthine (heteroxanthine), 3-methylxanthine; 1-methylxanthine, isobutylmethylxanthine (IBMX); 1-Hexyl-3,7-dimethylxanthine (pentifylline); and 1,7-dimethylxanthine. In some embodiments, the adenosine receptor antagonist is a xanthine compound, preferably a methylxanthine compound or a pharmaceutically acceptable form thereof. In some embodiments, the ophthalmic composition provided herein comprises a combination of adenosine receptor antagonists, such as a combination of xanthine compounds and/or methylxanthine compounds, or pharmaceutically acceptable forms thereof. In some embodiments, the adenosine receptor antagonist is caffeine or 7-methylxanthine, a related compound, or a pharmaceutically acceptable form thereof, such as for example, caffeine citrate. In some embodiments, the adenosine receptor antagonist is caffeine. In some embodiments, the adenosine receptor antagonist is caffeine citrate.

In some embodiments, the adenosine receptor antagonist present in the ophthalmic composition is an amount in the range of between approximately 0.05-10.0 w/v. %, of the ophthalmic composition, for example, between approximately 0.05-5.0 w/v %, between approximately 0.1-4.0 w/v %, between approximately 0.1-3.0 w/v %, between approximately 0.1-2.0 w/v %, between approximately 0.1-1.0 w/v %, between approximately 0.5-5.0 w/v %, between approximately 1.0-5.0 w/v %, between approximately 1.0-2.0 w/v %, between approximately 2.0-5.0 w/v %, between approximately 3.0-5.0 w/v %, between approximately 4.0-5.0 w/v %, between approximately 4.0-10.0 w/v %, between approximately 2.0-10.0 w/v %, between approximately 3.0-10.0 w/v %, between approximately 4.0-10.0 w/v %, between approximately 5.0-10.0 w/v %, between approximately 6.0-10.0 w/v %, between approximately 7.0-10.0 w/v %, or between approximately 4.0-6.0 w/v % of the ophthalmic composition. In some embodiments, the adenosine receptor antagonist present in the ophthalmic composition is an amount of at least 0.05 w/v %, at least 0.1 w/v %, at least 0.2 w/v %, at least 0.5 w/v %, at least 1.0 w/v %, at least 2 w/v %, at least 3 w/v %, at least 4 w/v %, at least 5 w/v %, at least 6 w/v %, at least 7 w/v %, at least 8 w/v %, or at least 9 w/v % of the ophthalmic composition. In some embodiments, the adenosine receptor antagonist present in the ophthalmic composition is an amount of no more than 0.05 w/v %, no more than 0.1 w/v %, no more than 0.2 w/v %, no more than 0.5 w/v %, no more than 1.0 w/v %, no more than 2 w/v %, no more than 3 w/v %, no more than 4 w/v %, no more than 5 w/v %, no more than 6 w/v %, no more than 7 w/v %, no more than 8 w/v %, no more than 9 w/v %, or no more than 10 w/v % of the ophthalmic composition. In some embodiments, the amount of the adenosine receptor antagonist is a therapeutically effective amount. In some embodiments, the adenosine receptor antagonist is in the form of a pharmaceutically acceptable salt. In such instances, the amount (or weight percent) of the adenosine receptor antagonist is based on the non-salt form. In some embodiments, the adenosine receptor antagonist is a xanthine compound or a methylxanthine compound as disclosed herein, such as Caffeine.

In some embodiments, the ophthalmic composition may comprise a combination of an adenosine receptor antagonist and a parasympathomimetic agent. In some embodiments, the adenosine receptor antagonist is a xanthine compound or a methylxanthine compound, or a pharmaceutically acceptable form thereof. In some embodiments, the adenosine receptor antagonist is caffeine or 7-methylxanthine, or a pharmaceutically acceptable form thereof, such as for example caffeine citrate. In some embodiments, the parasympathomimetic agent is a cholinergic agent or a nicotinic agent, or a pharmaceutically acceptable form thereof. In some embodiments, the ophthalmic composition comprises one or more adenosine receptor antagonists and one or more parasympathomimetic agents. In some embodiments, the parasympathomimetic agent, or one or more parasympathomimetic agents, is selected from the group consisting of neostigmine, cevimeline, pilocarpine, aceclidine, carbachol, methacholine, echothiophate, physostigmine, prostigmine, acetylcholine, or a pharmaceutically acceptable form thereof (such as for example, phospholine iodide or echothiophate iodide).

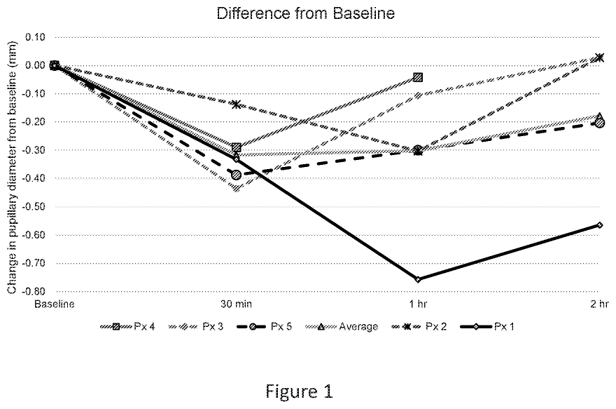

FIG. 1 illustrates the baseline pupillary diameter and the pupillary diameter over 2 hours in a group of 5 individuals (3 males and 2 females, age ranging from 31 to 45 yrs) prescribed with an ophthalmic composition comprising caffeine. A topical ophthalmic composition comprising caffeine 2 wt. % in water was instilled (as a single eye drop in the range of from 0.025 mL to 0.075 ml, such as an average single drop size of about 0.05 mL) in one eye of each of the individuals. Pupillary diameter at baseline and at subsequent time points was captured using a Grand Seiko autorefractor for 11 secs and data summarized. The room illumination was standardized at 150 to 200 lux. As observed from FIG. 1, there was a decrease in pupillary diameter post instillation. On average, compared to baseline, the decrease in pupillary diameter at 30 mins and 1 hr post instillation was 0.3 mm and was approximately 0.2 mm at 2 hrs. There was some variation between the various individuals, however the maximal decrease increase in pupillary diameter at 30 mins was 0.4 mm, was approximately 0.8 mm at 1 hr and was approximately 0.6 mm at 2 hrs. Such an ophthalmic composition, as disclosed herein, can be used according to the methods provided herein to reduce the pupillary diameter of a treated eye of a subject and be effective in treating or managing symptoms related to presbyopia and/or mydriasis and/or eyes with disorders requiring light modulation such as one or more of vision disturbances.

In some embodiments, a method of treating presbyopia and/or mydriasis and/or eyes with disorders requiring light modulation comprises administering to the eye and/or surrounding adnexa of an eye of an individual subject an ophthalmic composition comprising one or more of an adenosine receptor antagonist. In some embodiments, a method of treating presbyopia and/or mydriasis and/or eyes with disorders requiring light modulation comprises administering to the eye and/or surrounding adnexa of an eye of an individual subject an ophthalmic composition comprising one or more of an adenosine receptor antagonist and one or more of a parasympathomimetic agent. In some embodiments, the ophthalmic composition provided herein has a pH ranging from about 4.0 to about 8.0, such as about 4.0 to about 7.5, about 4.2 to about 7.5, or about 4.5 to about 7.5. In some embodiments, the adenosine receptor antagonist in the ophthalmic composition is a xanthine or a related compound. In some embodiments, the xanthine compound in the ophthalmic composition is caffeine or 7-methylxanthine, or a pharmaceutically acceptable salt thereof, for example, caffeine citrate.

In certain embodiments, the methods of treating provided herein, and/or use of the ophthalmic composition disclosed herein, reduces the pupil diameter of the eye of the individual and provides relief from symptoms or help reduce, mitigate or alleviate one or more symptoms associated with presbyopia and/or mydriasis and/or an ocular condition requiring modulation of light received at the retina of the eye of a subject. In certain embodiments, the methods of treating provided herein, and/or use of the ophthalmic composition disclosed herein, improves one or more parameters related to the visual performance of the eye of an individual subject, such as for example, the ability of the eye to see clearly at one or more distances or alleviate one or symptoms related to night vision disturbances. In certain embodiments, the methods of treating provided herein, and/or use of the ophthalmic composition disclosed herein, improves one or more parameters related to the visual performance of the eye of the individual subject by reducing the duration of symptoms experienced by the eye such as for example, symptoms related to night vision disturbances or may reduce the duration that the eye experiences blurred vision at one or more distances. In certain embodiments, the methods of treating provided herein, and/or use of the ophthalmic composition disclosed herein, provides an easier method of managing the condition or disorder of the eye by administration of the drop, i.e., dosing the eye with a drop compared to management with spectacles or contact lenses that require constant wear and care regimen.

In some embodiments, the method of treating provided herein comprises administering the ophthalmic composition disclosed herein to the eye and/or surrounding adnexa of at least one eye of a subject.

In some embodiments, the concentration of the adenosine receptor antagonist may range from about 0.5% to about 10.0 w/v %, relative to the ophthalmic composition. In some embodiments, the concentration of the adenosine receptor antagonist, for example a xanthine compound or a methylxanthine compound, such as caffeine or a pharmaceutically acceptable form thereof, may range from between approximately 0.05 w/v % to about 10.0 w/v % relative to the ophthalmic composition. In other embodiments, the concentration of the adenosine receptor antagonist, for example a xanthine compound or a methylxanthine compound, such as caffeine or a related compound, may range from between approximately 0.05 w/v % to about 8.0 w/v %, between approximately 0.5 w/v % to about 7.0 w/v %, between approximately 0.5 w/v % to about 5.0 w/v %, between approximately 1.0 w/v % to 6.0 w/v %, between approximately 2.0 w/v % to 6.0 w/v %, between approximately 3.0 w/v % to 6.0 w/v %, or between approximately 4.0 w/v % to 6.0 w/v %, relative to the ophthalmic composition. In some embodiments, the concentration of the adenosine receptor antagonist, for example a xanthine compound or a methylxanthine compound, such as caffeine or a related compound, is approximately 1.0 w/v %, approximately 2.0 w/v %, approximately 3.0 w/v %, approximately 4.0 w/v %, approximately 5.0 w/v %, approximately 6.0 w/v %, approximately 7.0 w/v %, or approximately 8.0 w/v %, relative to the ophthalmic composition. In some embodiments, the concentration of the adenosine receptor antagonist, for example a xanthine compound or a methylxanthine compound, such as caffeine or a related compound, is at least 0.05 w/v %, at least 0.1 w/v %, at least 0.2 w/v %, at least 0.5 w/v %, at least 1.0 w/v %, at least 2 w/v %, at least 3 w/v %, at least 4 w/v %, at least 5 w/v %, at least 6 w/v %, at least 7 w/v %, at least 8 w/v %, or at least 9 w/v % relative to the ophthalmic composition. In some embodiments, the concentration of the adenosine receptor antagonist, for example a xanthine compound or a methylxanthine compound, such as caffeine or a related compound, is present in amount of no more than 0.05 w/v %, no more than 0.1 w/v %, no more than 0.2 w/v %, no more than 0.5 w/v %, no more than 1.0 w/v %, no more than 2 w/v %, no more than 3 w/v %, no more than 4 w/v %, no more than 5 w/v %, no more than 6 w/v %, no more than 7 w/v %, no more than 8 w/v %, no more than 9 w/v %, or no more than 10 w/v %, relative to the ophthalmic composition. In other embodiments, the concentration of the adenosine receptor antagonist, for example a xanthine compound or a methylxanthine compound, such as caffeine or a related compound, or a pharmaceutically acceptable thereof, may range from about 0.5 g per 100 mL to 20 g per 100 mL of ophthalmic composition, such as about 0.5 g to 15 g per 100 mL, about 0.5 g to 10 g per 100 mL, about 0.5 g to about 5 g per 100 mL, about 0.5 g/100 mL, about 1.0 g/100 mL, about 1.5 g/100 mL, about 2.0 g/100 mL, about 3.0 g/100 mL, about 4.0 g/100 mL, about 5.0 g/100 mL, about 5 g to about 10 g/100 mL, about 2.0 g to about 5 g/100 mL, or about 5 g to about 15 g/100 mL of ophthalmic composition.

In some embodiments, the ophthalmic composition disclosed herein comprises an adenosine receptor antagonist, or a pharmaceutically acceptable form thereof, and optionally further comprises a parasympathomimetic agent, or a pharmaceutically acceptable form thereof.

In some embodiments, the ophthalmic composition disclosed herein comprises an adenosine receptor antagonist, or a pharmaceutically acceptable form thereof, and optionally further comprises a parasympathomimetic agent, a muscarinic receptor agonist, an alpha-adrenergic antagonist, a vasoconstricting agent, an anti-inflammatory agent, or an astringent, or a pharmaceutically acceptable form thereof.

In some embodiments, the ophthalmic composition comprising the adenosine receptor antagonist further comprises an anti-inflammatory agent or an astringent. In some embodiments, the ophthalmic composition comprising the adenosine receptor antagonist further comprises a vaso-constricting agent. In some embodiments, the anti-inflammatory or an astringent is zinc sulfate, zinc lactate, allantoin, epsilon-aminocaproic acid, indomethacin, lysozyme chloride, silver nitrate, pranoprofen, sodium azulenesulfonate, dipotassium glycyrrhizinate, diammonium glycyrrhizinate, Diclofenac sodium, bromfenac sodium, nepafenac, ketorolac, cyclosporine, flurbiprofen, berberine chloride, or berberine sulfate, or a combination thereof.

In some embodiments, the method of treating provided herein and/or the use of the ophthalmic composition disclosed herein reduces the pupillary diameter of the eye of an individual subject by approximately 0.1 mm to about 3.5 mms. In some embodiments, the reduction in pupillary diameter of the eye of an individual subject using treated according to the method of treating provided herein and/or using the ophthalmic composition disclosed herein ranges from about 0.2 mm to about 2 mms, about 0.3 mm to about 2 mms, about 0.4 mms to about 2 mms, about 0.5 mm to about 2 mm, about 0.6 mm to about 2 mms, about 0.7 mm to about 2 mm, about 0.8 mm to about 2 mm, about 0.9 mm to about 2 mm, about 1 mm to about 2 mm, about 1.1 mm to about 2 mm, about 1.2 mm to about 2 mm, about 1.3 mm to about 2 mm, about 1.4 mm to about 2 mm, about 1.5 mm to about 2 mm, about 0.2 mm to about 1 mm, about 0.5 mm to about 1 mm, about 0.5 mm to about 3 mm, about 0.5 mm to about 3.5 mm, about 1 mm to about 3 mm, about 1.5 mm to about 3.0 mm, about 2.0 mm to about 3.0 mm, about 2.5 mm to about 3.0 mm, or about 3.0 mm to about 3.5 mm. In some embodiments, the reduction in pupillary diameter of the eye according to the method of treating provided herein and/or the use of the ophthalmic composition disclosed herein is about 5%, about 10%, about 15%, about 20%, about 25%, about 30%, about 35%, about 40%, about 45%, or about 50%, relative to the pupillary diameter at baseline.

In some embodiments, the method of treating provided herein and/or the use of the ophthalmic composition disclosed herein improves at least one of the photopic and/or mesopic distance visual acuity, intermediate visual acuity or near visual acuity achieved by the treated subject by approximately 1 line. In some embodiments, the improvement at least one of the photopic and/or mesopic distance visual acuity, intermediate visual acuity or near visual acuity achieved by the treated subject is in the range from about 0.02 log MAR (logarithm of the minimum angle of resolution) to about 0.5 log MAR, about 0.02 log MAR to about 0.4 log MAR, 0.02 log MAR to about 0.3 log MAR, or 0.02 log MAR to about 0.1 log MAR. In some embodiments, the improvement at least one of the photopic and/or mesopic distance visual acuity, intermediate visual acuity or near visual acuity achieved by the treated subject is about 0.02 log MAR, about 0.1 log MAR, about 0.06 log MAR, about 0.14 log MAR, about 0.2 log MAR, about 0.24 log MAR, or about 0.3 log MAR.

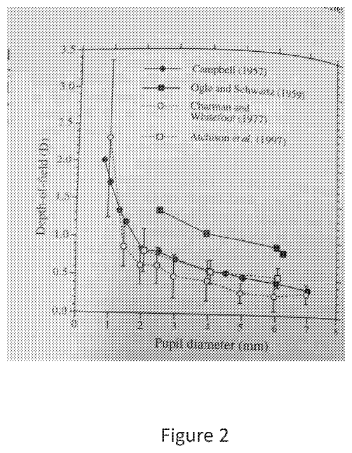

In some embodiments, the method of treating provided herein and/or the use of the ophthalmic composition disclosed herein improves the depth of the field of the treated eye. It has been indicated that increasing the depth of field may be advantageous for example where age related reduction in amplitude of accommodation can be improved by increasing depth of field (see, e.g., FIG. 2). An increased depth of field increases the range over which there is acceptable vision. In some embodiments, the method of treating provided herein and/or the use of the ophthalmic composition disclosed herein increases the depth of field of the treated eye by about 0.25D to about 2.5D, for example, increases the depth of field of the treated eye by about 0.25D, about 0.5D, about 0.75D, about 1D, about 1.25D, about 1.5D, about 1.75D, about 2D and about 2.25D.

In some embodiments, the method of treating provided herein and/or the use of the ophthalmic composition disclosed herein improves the depth of the field of the treated eye and therefore improves the range of clear vision for near distances.

In some embodiments, the method of treating provided herein and/or the use of the ophthalmic composition disclosed herein reduces the duration of symptoms experienced by the individual subject. In some embodiments, the method of treating provided herein and/or the use of the ophthalmic composition disclosed herein reduces the duration of symptoms experienced by the individual subject by about 0.5 hour to about 5 hours, such as by about 0.5 hour to about 4 hours, by about 0.5 hour to about 3 hours, by about 0.5 hour to about 2.5 hours, by about 0.5 hours to about 2 hours, by about 0.5 hours to about 1.5 hours, or by about 0.5 hours to about 1.0 hours.

In some embodiments, the ophthalmic composition disclosed herein and the methods of treating provided herein comprising administering the same may be delivered to the eye of the subject and/or surrounding adnexa in any suitable form. In some embodiments, the forms of delivery of the ophthalmic composition disclosed herein may be delivered as an aqueous formulation, suspension, topical ointment, surface ointment or cream, gel, spray, an extended release system or an encapsulation such as those utilizing liposomes, nanosomes, nanoparticles, encapsulation, a polymeric matrix systems such as hydrogels, contact lenses, biodegradable materials, molecular printing, an ocular insert or a combination of one or more of the above. In some embodiments, the ophthalmic composition is delivered as an eye drop aqueous formulation. In some embodiments, the ophthalmic composition is delivered via an extended delivery system.

In certain embodiments, the ophthalmic composition disclosed herein (or the ophthalmic device comprising the same, or the method of treating using the same), further comprises one or more additional ophthalmically acceptable excipients and additives, comprising carriers, stabilizers, an osmolarity adjusting agent, a preservative, a buffer agent, a tonicity adjusting agent, thickeners, or other excipients.

In some embodiments, the ophthalmic composition disclosed herein comprises a carrier. Suitable carries include one or more of the following: water; mixtures of water and water-miscible solvents, such as C₁- to C₇-alkanols; vegetable oils or mineral oils comprising from 0.5 to 5% non-toxic water-soluble polymers; gelling products, such as gelatin, alginates, pectins, tragacanth, karaya gum, xanthan gum, carrageenan, agar and acacia, and their derivatives; starch derivatives, such as starch acetate and hydroxypropyl starch; cellulose and its derivatives and also other synthetic products, such as polyvinyl alcohol, polyvinylpyrrolidone, polyvinyl methyl ether, polyethylene oxide, cross-linked polyacrylic acid, such as neutral Carbopol. or mixtures of those polymers or any other carrier known to those skilled in the art.

In some embodiments, the ophthalmic composition disclosed herein comprises an osmolarity adjusting agent. Suitable osmolarity adjusting agents include sodium chloride, or any other osmolarity adjusting agent known to those skilled in the art.

In some embodiments, the ophthalmic composition disclosed herein comprises a preservative. Suitable preservatives include one or more of polydronium chloride, alkyldiaminoethylglycine hydrochloride, sodium benzoate, ethanol, benzalkonium chloride, benzethonium chloride, chlorhexidine gluconate, chlorobutanol, sorbic acid, potassium sorbate, dehydroacetic acid Sodium, methyl paraoxybenzoate, ethyl paraoxybenzoate, propyl paraoxybenzoate, butyl paraoxybenzoate, oxyquinoline sulfate, phenethyl alcohol, benzyl alcohol, biguanide compound or a combination thereof or any other preservative known to those skilled in the art.

In some embodiments, the ophthalmic composition disclosed herein comprises a buffer agent. For the adjustment of the pH of the ophthalmic composition, for example, to a physiological pH, inclusion of buffer agents may be useful. In some embodiments, the ophthalmic composition is an aqueous formulation. The pH of the aqueous formulation is typically be maintained within the range of between about 4.0 to 8.0. Other ranges may also be used, for example, between about 4.0 to 7.5, between about 4.0 to 7.0, between about 4.0 to 6.5, between about 4.5 to 8.0, between about 5.0 to 8.0, between about 5.5 to 8.0, between about 6.0 to 8.0, between about 6.5 to 8.0, between about 7.0 to 8.0, between about 5.2 to 7.5, between about 5.5 to 7.5, between about 6.0 to 7.5, between about 6.5 to 7.5, between about 6.8 to 7.2, or between about 7.3 to 7.5. Selecting a pH or pH range that is compatible, or substantially compatible, with the ocular surface is typically desired. Suitable buffer agents may be selected from: borates, borate-polyol complexes, succinate, phosphate buffering agents, citrate buffering agents, acetate buffering agents, carbonate buffering agents, organic buffering agents, acetate buffering agents, aminoacid buffering agents boric acid; sodium borate; potassium citrate; citric acid; sodium bicarbonate; and TRIS, disodium edetate (EDTA) and various phosphate buffers (including combinations of NaCl, KCl, Na₂HPO₄, NaH₂PO₄ and KH₂PO₄) and mixtures thereof. In certain embodiments, the buffer used contains concentrations of about NaCl 8 g/L, KCl 0.2 g/L, Na₂HPO₄ 1.15 g/L and KH₂PO₄ 0.2 g/L.

In some embodiments, the ophthalmic composition disclosed herein comprises a tonicity adjusting agent. Suitable tonicity adjusting agents may, for example, be of ionic and/or non-ionic type. In some embodiments, the tonicity adjusting agent is selected from sodium chloride, sodium nitrate, sodium sulfate, sodium bisulfate, potassium chloride, calcium chloride, magnesium chloride, zinc chloride, potassium acetate, sodium acetate, sodium bicarbonate, sodium carbonate, sodium thiosulfate, magnesium sulfate, disodium hydrogen phosphate, sodium dihydrogen phosphate, potassium dihydrogen phosphate, dextrose, mannitol, sorbitol, dextrose, sucrose, urea, propylene glycol, glycerin, trehalose, or a combination thereof.

In some embodiments, the ophthalmic composition disclosed herein comprises a thickener. Thickeners that may be suitable include methylcellulose (MC), hydroxyethylcellulose (HEC), hydroxypropylcellulose (HPC), hydroxypropyl-methylcellulose (HPMC), sodium carboxymethylcellulose (NaCMC), and other swellable hydrophilic polymers such as polyvinyl alcohol (PVA), hyaluronic acid or a salt thereof (e.g., sodium hyaluronate), crosslinked acrylic acid polymers or “carbomers” or any combination thereof or other thickeners that will be known to those of ordinary skilled in the art. The preferred amount of any thickener is such that it is comfortable for the eye and allows retention of the formulation in the eye.

The dosage form of the ophthalmic composition may be determined by the route of administration. An effective amount of the adenosine receptor antagonist, or more specifically a xanthine compound, such as caffeine, may be administered as a single dose, multiple doses during the day, or provided as an extended release system.

### EXAMPLES

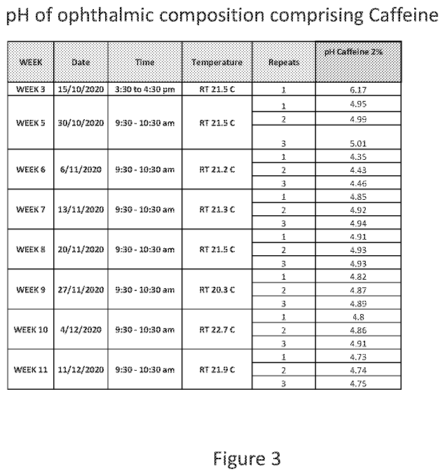

FIG. 3 provides the pH of an ophthalmic composition comprising caffeine at 2.0 w/v. % of the formulation in water over an extended period. Caffeine was formulated as unit dose solution in water and stored in a refrigerator at about 3 to 7° C. in a plastic vial. As seen from the table, the pH varies from about 4.4 to about 6.2.

### Example 1

The following Table 1 and Table 2 provide examples of ophthalmic compositions according to certain embodiments. The ophthalmic composition comprises a xanthine compound, such as caffeine or a related compound, with or without a buffer, a tonicity agent and a presentative.

TABLE 1
Composition 1: Topical aqueous composition
(eye drop)- unit dose preparation- 100 mL

| Ingredient | Concentration (w/v %) |
|---|---|
| Caffeine | 2 |
| Sodium chloride 0.9 wt. % solution | 98 |
| pH | 4.7 to 4.9 |

TABLE 2
Composition 2: Topical aqueous composition (eye drop)- 100 mL

| Ingredient | Concentration (w/v %) |
|---|---|
| Caffeine | 2 |
| Sorbitan monoleate | 0.05 |
| Potassium dihydrogen orthophosphate | 0.2 |
| (KH₂PO₄₎ | |
| Sodium Chloride 0.9 wt. % Solution | 97.75 |

### Example 2—Use of an Aqueous Formulation Comprising Caffeine for Reversal of Mydriasis

The following Table 3 illustrates the pupil diameter of a 53 yr old female. An aqueous solution of 1% Tropicamide was instilled in both eyes. Pupillary diameter was measured over a 15 second interval with a Grand Seiko autorefractor with the individual fixating at an object positioned 1 meter away. Pupillary diameter was dynamically sampled at multiple time points over the 15 second interval. Pupillary diameters for both eyes were measured prior to instillation (baseline), soon after instillation of 1% Tropicamide and then 20 minutes and 50 minutes post instillation. As shown in Table 3, there was a difference of approximately 0.1 mm at baseline between the eyes, and after instillation of Tropicamide 1%, at 50 minutes, there was a difference of 0.3 mm. At this time point, a single drop (0.05 mL) of 2.0 w/v % Caffeine in Saline (0.9 wt. % NaCl) (composition 1) was instilled in the left eye only. The individual was monitored 30 minutes, 60 minutes, 2 hrs, 4 hrs and 6.5 hrs from instillation of Caffeine. As seen from the table, following instillation of Caffeine in the left eye, at 1 and 2 hours, the difference between the pupils was approximately 0.8 mm with the left eye exhibiting a reversal in pupil diameter whereas the right eye continued to exhibit a large pupil.

TABLE 3

| | | Pupillary | | Difference |
|---|---|---|---|---|
| | | diameter (mm) | | in pupillary |
| | | Right | Left | diameter (mm) |
| Time | Activity | eye | eye | (Right − Left) |
| 14.20 | Baseline | 3.6 | 3.5 | 0.1 |
| 14.24 | Post Instillation -Tropicamide | 3.7 | 3.5 | 0.2 |
| | 1% | | | |
| 14.44 | 20 mins Post Instillation | 7.2 | 6.8 | 0.4 |
| 15.14 | 50 mins Post Instillation | 7.2 | 6.9 | 0.3 |
| 15.14 | Instillation of Caffeine 2 w/v % | — | — | — |
| | in left eye | | | |
| 15.44 | 30 mins post instillation of | 7.3 | 6.8 | 0.5 |
| | Caffeine 2 w/v % | | | |
| 16.20 | 60 mins post instillation of | 7.2 | 6.5 | 0.7 |
| | Caffeine 2 w/v % | | | |
| 17.20 | 2 hrs post instillation of | 7.1 | 6.3 | 0.8 |
| | Caffeine 2 w/v % | | | |

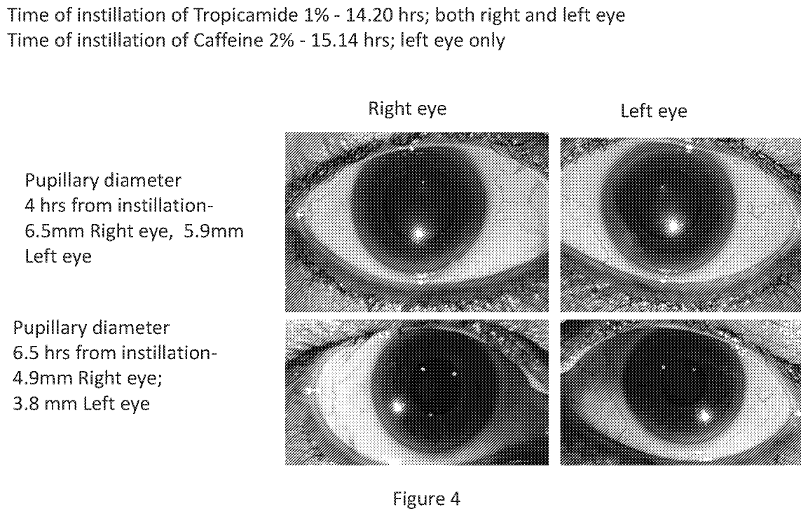

FIG. 4 illustrates the difference in pupillary diameter between the right and left eyes 4 hrs from the instillation of Tropicamide 1% in both eyes and 3 hrs from instillation of Caffeine 2 w/v % in the left eye. As seen from the top panel, the right eye pupillary diameter is larger at 6.5 mm versus 5.9 mm in the left eye. At 6.5 hrs from the baseline visit, the left eye was observed to have returned to normal with a pupil diameter of 3.8 mm whereas the right eye pupil was still enlarged at 4.9 mm.

### Exemplary Embodiments

One or more than one (including for instance all) of the following exemplary Embodiments may comprise each of the other embodiments or parts thereof.

A1. An ophthalmic composition comprising: (i) an adenosine receptor antagonist or a pharmaceutically acceptable salt thereof, (11) optionally one or more parasympathomimetics and/or muscarinic receptor agonists; and (iii) a pharmaceutically acceptable excipient or carrier.

A2. The ophthalmic composition of Embodiment A1, wherein the adenosine receptor antagonist is a xanthine compound, or pharmaceutically acceptable salt thereof.

A3. The ophthalmic composition of Embodiment A1 or Embodiment A2, wherein the adenosine receptor antagonist is methylxanthine compound, or pharmaceutically acceptable salt thereof.

A4. The ophthalmic composition of any one of Embodiments A1-A3, wherein the adenosine receptor antagonist or pharmaceutically acceptable salt thereof is selected from the group consisting of caffeine (1,7 methylxanthine), 7-methylxanthine, 1,7-dimethylxanthine (paraxanthine), 3,7-dimethylxanthine (theobromine); 7-methylxanthine (heteroxanthine), 3-methylxanthine, 1-methylxanthine, isobutylmethylxanthine (IBMX), 1-Hexyl-3,7-dimethylxanthine (pentifylline), or 1,7-dimethylxanthine, or a pharmaceutically acceptable salt thereof.

A5. The ophthalmic composition of any one of Embodiments A1-A4, wherein the adenosine receptor antagonist is caffeine (1,7 methylxanthine).

A6. The ophthalmic composition of any one of Embodiments A1-A5, wherein the adenosine receptor antagonist is caffeine citrate.

A7. The ophthalmic composition of Embodiment A1, wherein the adenosine receptor antagonist or pharmaceutically acceptable salt thereof is non-selective adenosine receptor antagonist.

A8. The ophthalmic composition of Embodiment A7, wherein the non-selective antagonist acts on one or more of adenosine receptors subtypes of A1, A2a, A2b or A3.

A9. The ophthalmic composition of any one of Embodiments A1-A8, wherein the adenosine receptor antagonist or pharmaceutically acceptable salt thereof is present in an amount in the range of from about 0.05 w/v % to about 10 w/v % of the ophthalmic composition, such as in a therapeutically effective amount.

A10. The ophthalmic composition of Embodiment A9, wherein the adenosine receptor antagonist or pharmaceutically acceptable salt thereof is present in an amount in the range of between approximately 0.05-5.0 w/v %, 0.1-4.0 w/v %, between approximately 0.1-3.0 w/v %, between approximately 0.1-2.0 w/v %, between approximately 0.1-1.0 w/v %, between approximately 0.5-5.0 w/v %, between approximately 1.0-5.0 w/v %, between approximately 1.0-2.0 w/v %, between approximately 2.0-5.0 w/v %, between approximately 3.0-5.0 w/v %, between approximately 4.0-5.0 w/v %, between approximately 4.0-10.0 w/v %, between approximately 2.0-10.0 w/v % between approximately 3.0-10.0 w/v %, between approximately 4.0-10.0 w/v %, between approximately 5.0-10.0 w/v %, between approximately 6.0-10.0 w/v %, between approximately 7.0-10.0 w/v %, or between approximately 4.0-6.0 w/v % of the ophthalmic composition.

All. The ophthalmic composition of any one of Embodiments A1-A10, wherein the ophthalmic composition comprises one or more parasympathomimetics and/or muscarinic receptor agonists.

A12. The ophthalmic composition of any one of Embodiments A1-A11, wherein the parasympathetic agent is a cholinergic agent or a nicotinic agent.

A13. The ophthalmic composition of any one of Embodiments A1-A12, wherein the parasympathomimetic agent is selected from the group consisting of neostigmine, cevimeline, pilocarpine, aceclidine, carbachol, methacholine, echothiophate, physostigmine, prostigmine, phospholine iodide, demecarium bromide, and related compounds.

A14. The ophthalmic composition of any one of Embodiments A1-A13, wherein the ophthalmic composition further comprises an alpha-adrenergic antagonist, a muscarinic receptor agonist, a vasoconstricting agent, an anti-inflammatory agent, or an astringent, or a pharmaceutically acceptable form thereof, or combinations thereof.

A15. The ophthalmic composition of any one of Embodiments A1-A14, wherein the ophthalmic composition further comprises an alpha-adrenergic antagonist, or a pharmaceutically acceptable form thereof.

A16. The ophthalmic composition of any one of Embodiments A1-A15, wherein the ophthalmic composition further comprises a muscarinic receptor agonist, or a pharmaceutically acceptable form thereof.

A17. The ophthalmic composition of any one of Embodiments A1-A16, wherein the ophthalmic composition further comprises a vasoconstricting agent, or a pharmaceutically acceptable form thereof.

A18. The ophthalmic composition of any one of Embodiments A1-A17, wherein the ophthalmic composition further comprises an anti-inflammatory agent, or a pharmaceutically acceptable form thereof.

A19. The ophthalmic composition of any one of Embodiments A1-A18, wherein the ophthalmic composition further comprises an astringent, or a pharmaceutically acceptable form thereof.

A20. The ophthalmic composition of any one of Embodiments A1-A19, wherein the ophthalmic composition further comprises one or more additional ophthalmically acceptable excipients and/or additives.

A21. The ophthalmic composition of any one of Embodiments A1-A20, wherein the ophthalmic composition further comprises a carrier, a stabilizer, an osmolarity adjusting agent, a preservative, a buffer agent, a tonicity adjusting agent, a thickener, or other excipient.

A22. The ophthalmic composition of any one of Embodiments A1-A21, wherein the ophthalmic composition has a pH in the range of physiological pH or compatible, or substantially compatible, with the ocular surface is typically desired.

A23. The ophthalmic composition of any one of Embodiments A1-A22, wherein the ophthalmic composition has a pH in the range of about 4.0 to about 8.0.

A24. The ophthalmic composition of any one of Embodiments A1-A23, wherein the ophthalmic composition has a pH in the range of between about 4.0 to 7.5, between about 4.0 to 7.0, between about 4.0 to 6.5, between about 4.5 to 8.0, between about 5.0 to 8.0, between about 5.5 to 8.0, between about 6.0 to 8.0, between about 6.5 to 8.0, between about 7.0 to 8.0, between about 5.2 to 7.5, between about 5.5 to 7.5, between about 6.0 to 7.5, between about 6.5 to 7.5, between about 6.8 to 7.2, or between about 7.3 to 7.5.

A25. The ophthalmic composition of any one of Embodiments A1-A24, wherein the ophthalmic composition is in the form of a solution, a suspension, an ointment, a cream, a gel, a spray, an extended-release system such as those utilizing liposomes, niosomes, nanoparticles, encapsulation, a polymeric matrix systems such as hydrogels, a contact lens, a biodegradable material, a molecular printing, or an ocular insert.

A26. The ophthalmic composition of any one of Embodiments A1-A25, wherein the ophthalmic composition is in the form of an aqueous solution.

A27. The ophthalmic composition of any one of Embodiments A1-A26, wherein the ophthalmic composition is in the form of an aqueous eye drop solution.

A28. The ophthalmic composition of any one of Embodiments A1-A27, wherein the ophthalmic composition is suitable for delivery to the eye and/or the surrounding adnexa of the eye of a subject.

B1. An ophthalmic delivery system comprising the ophthalmic composition of any one of Embodiments A1-A28 for delivery to the eye and/or the surrounding adnexa of the eye of a subject.

C1. A method of treating presbyopia in a subject, the method comprising administering the ophthalmic composition of any one of Embodiments A1-A28 to the eye and/or the surrounding adnexa of the eye of the subject or utilizing the ophthalmic delivery system of Embodiments B1.

C2. A method of treating mydriasis in a subject, the method comprising administering the ophthalmic composition of any one of Embodiments A1-A28 to the eye and/or the surrounding adnexa of the eye of the subject.

C3. A method of treating a disorder of the eye requiring modulation of light received at the eye in a subject, the method comprising administering the ophthalmic composition of any one of Embodiments A1-A28 to the eye and/or the surrounding adnexa of the eye of the subject.

C4. A method of managing or treating an eye of a subject following refractive surgery or a distorted, scarred or astigmatic cornea, the method comprising administering the ophthalmic composition of any one of Embodiments A1-A28 to the eye of the subject.

C5. The method of any one of Embodiments C1-C4, wherein the method further comprises administering either simultaneously or sequentially to the eye of the subject a mydriatic or a cycloplegic agent.

C6. The method of any one of Embodiments C1-C5, wherein the method reduces one or more side effects related to the disorder of the eye.

C7. The method of any one of Embodiments C1-C6, wherein the method improves the performance of one or more visual performance measures in the at least one eye of the subject.

C8. The method of Embodiment C7, wherein the one or more visual performance measures is selected from the group consisting of high or low contrast distance visual acuity, high or low contrast intermediate visual acuity, high or low contrast near visual acuity, haloes, and glare and night vision.

C9. The method of any one of Embodiments C1-C8, wherein the method reduces the pupillary diameter of the eye of the subject.

C10. The method of any one of Embodiments C1-C9, wherein the method reduces the pupillary diameter of the eye of the subject by approximately 0.1 mm to about 3.5 mms.

C11. The method of any one of Embodiments C1-C10, wherein the method reduces the pupillary diameter of the eye of the subject by approximately 0.2 mm to about 2 mms.

C12. The method of any one of Embodiments C9-C11, wherein the reduction in pupillary diameter of the eye of the subject ranges from about 0.1 mm to about 3.5 mms, about 0.3 mm to about 3 mms, about 0.4 mms to about 3 mms, about 0.5 mm to about 3 mm, about 0.6 mm to about 3 mms, about 0.7 mm to about 3 mm, about 0.8 mm to about 3 mm, about 0.9 mm to about 3 mm, about 1 mm to about 3 mm, about 1.1 mm to about 3 mm, about 1.2 mm to about 3 mm, about 1.3 mm to about 3 mm, about 1.4 mm to about 3 mm, about 1.5 mm to about 3 mm, about 0.2 mm to about 1 mm, about 0.5 mm to about 1 mm, about 0.5 mm to about 3 mm, about 0.5 mm to about 3.5 mm, about 1 mm to about 3 mm, about 1.5 mm to about 3.0 mm, about 2.0 mm to about 3.0 mm, about 2.5 mm to about 3.0 mm, or about 3.0 mm to about 3.5 mm.

C13. The method of any one of Embodiments C9-C12, wherein the reduction in pupillary diameter of the eye of the subject is about 5%, about 10%, about 15%, about 20%, about 25%, about 30%, about 35%, about 40%, about 45%, or about 50%, relative to the pupillary diameter at baseline.

C14. The method of any one of Embodiments C1-C13, wherein the method provides relief from symptoms or help reduce, mitigate or alleviate one or more symptoms associated with presbyopia and/or mydriasis and/or an ocular condition requiring modulation of light received at the retina of the eye of said subject.

C15. The method of any one of Embodiments C1-C14, wherein the method improves one or more parameters related to the visual performance of the eye of the subject.

C16. The method of Embodiment C15, wherein the one or more parameters related to the visual performance comprises an ability of the eye to see clearly at one or more distances or alleviate one or symptoms related to night vision disturbances.

C17. The method of any one of Embodiments C1-C16, wherein the method reduces the duration of one or more symptoms experienced by the eye of the subject.

C18. The method of Embodiment C16, wherein the one or more symptoms experienced by the eye of the subject is night vision disturbances or blurred vision at one or more distances.

C19. The method of any one of Embodiments C1-C18, wherein the method reduces the duration of symptoms experienced by the eye of the subject by about 0.5 hour to about 5 hours.

C20. The method of any one of Embodiments C1-C19, wherein the method reduces the duration of symptoms experienced by the eye of the subject by about 0.5 hour to about 4 hours, by about 0.5 hour to about 3 hours, by about 0.5 hour to about 2.5 hours, by about 0.5 hours to about 2 hours, by about 0.5 hours to about 1.5 hours, or by about 0.5 hours to about 1.0 hours.

C21. The method of any one of Embodiments C1-C20, wherein the method provides an easier method of managing the condition or disorder of the eye by administration of the ophthalmic composition.

C22. The method of any one of Embodiments C1-C21, wherein the method improves at least one of the photopic and/or mesopic distance visual acuity, intermediate visual acuity or near visual acuity achieved by the treated subject by approximately 1 line.

C23. The method of any one of Embodiments C1-C22, wherein the improvement at least one of the photopic and/or mesopic distance visual acuity, intermediate visual acuity or near visual acuity achieved by the treated subject is in the range from about 0.02 log MAR (logarithm of the minimum angle of resolution) to about 0.5 log MAR, about 0.02 log MAR to about 0.4 log MAR, 0.02 log MAR to about 0.3 log MAR, 0.02 log MAR to about 0.1 log MAR; or is about 0.02 log MAR, about 0.1 log MAR, about 0.06 log MAR, about 0.14 log MAR, about 0.2 log MAR, about 0.24 log MAR, or about 0.3 log MAR.

C24. The method of any one of Embodiments C1-C23, wherein the method improves the depth of the field of the treated eye.

C25. The method of any one of Embodiments C1-C24, wherein the method improves the depth of the field of the treated eye and improves the range of clear vision for near distances.

C26. The method of any one of Embodiments C1-C25, wherein the method increases the depth of field of the treated eye by about 0.25D to about 2.0D.

C27. The method of any one of Embodiments C1-C26, wherein the method increases the depth of field of the treated eye by about 0.25D to about 2.5D.

C28. The method of any one of Embodiments C1-C27, wherein the method increases the depth of field of the treated eye by about 0.25D, about 0.5D, about 0.75D, about 1D, about 1.25D, about 1.5D, about 1.75D, about 2.0D, or about 2.25D.

C29. The method of any one of Embodiments C1-C28, wherein prevents, controls, slows, reduces, retards, and/or mitigates the near vision related deficits that accompany presbyopia and/or mydriasis.

C30. The method of any one of Embodiments C1-C29, wherein the subject has presbyopia.

C31. The method of any one of Embodiments C1-C30, wherein the subject has mydriasis.

C32. The method of any one of Embodiments C1-C31, wherein the subject has a disorder of the eye requiring modulation of light received at the eye.

C33. The method of any one of Embodiments C1-C32, wherein the subject had refractive surgery or has a distorted, scarred or astigmatic cornea.

C34. The method of any one of Embodiments C1-C33, wherein the ophthalmic composition of any one of Embodiments A1-A28 is delivered via an extended delivery system.

It will be understood that the embodiments disclosed and defined in this specification extends to all alternative combinations of two or more of the individual features mentioned or evident from the text or drawings. All these different combinations constitute various alternative aspects of the present disclosure.

The foregoing outlines features of several embodiments so that those skilled in the art may better understand the aspects of the present disclosure. Those skilled in the art should appreciate that they may readily use the present disclosure as a basis for designing or modifying other processes and structures for carrying out the same purposes and/or achieving the same advantages of the embodiments introduced herein. Those skilled in the art should also realize that such equivalent constructions do not depart from the spirit and scope of the present disclosure, and that they may make various changes, substitutions, and alterations herein without departing from the spirit and scope of the present disclosure.

1. A ophthalmic composition comprising:
an adenosine receptor antagonist (e.g., caffeine or a related compound), or a pharmaceutically acceptable salt thereof, and optionally one or more parasympathomimetics and/or muscarinic receptor agonists; and
a pharmaceutically acceptable excipient or carrier.

2. The ophthalmic composition of claim 1, wherein the ophthalmic composition is used to reduce the pupillary diameter of an eye with presbyopia and/or mydriasis.

3. The ophthalmic composition of claim 1, wherein the ophthalmic composition is used to prevent, control, slow, reduce, retard, and/or mitigate the near vision related deficits that accompany presbyopia and/or mydriasis.

4. The ophthalmic composition of claim 1, wherein the ophthalmic composition is suited for delivery to the eye and/or the surrounding adnexa of the eye.

5. The ophthalmic composition of claim 1, wherein the non-selective adenosine receptor antagonist comprises xanthine related compounds comprising any combination of one or more of caffeine (1,7 methylxanthine), 7-methylxanthine; 1,7-dimethylxanthine (paraxanthine), 3,7-dimethylxanthine (theobromine); 7-methylxanthine (heteroxanthine), 3-methylxanthine; 1-methylxanthine, isobutylmethylxanthine (IBMX); 1-Hexyl-3,7-dimethylxanthine (pentifylline); and 1,7-dimethylxanthine.

6. The ophthalmic composition of claim 1, wherein the parasympathomimetic agent comprises any combination of one or more of neostigmine, cevimeline, pilocarpine, aceclidine, carbachol, methacholine, echothiophate, physostigmine, prostigmine, phospholine iodide, demecarium bromide and related compounds.

7. The ophthalmic composition of claim 1, wherein the concentration of caffeine or related compound may range from about 0.5 w/v % to about 10 w/v % of the composition.

8. The ophthalmic composition of claim 1, wherein the ophthalmic composition comprises a therapeutically effective amount of an adenosine receptor antagonist (e.g., caffeine and/or related compound) at about 0.5 w/v % to about 10 w/v % of the composition and further comprises any combination of one of more of a parasympathomimetic agent, alpha-adrenergic antagonist, muscarinic receptor agonist, a vasoconstricting agent or an anti-inflammatory agent.

9. The ophthalmic composition of claim 1, wherein the ophthalmic composition has a pH in the range of about 4 to about 7.5.

10. The ophthalmic composition of claim 1, wherein the ophthalmic composition reduces the effective pupillary diameter of the eye by approximately 0.2 mm to about 2 mms.

11. The ophthalmic composition of claim 1, wherein the adenosine receptor antagonist is a non-selective antagonist acting on one or more of adenosine receptors subtypes of A1, A2a, A2b or A3.

12. The ophthalmic composition of claim 1, wherein the adenosine receptor antagonist is a xanthine compound.

13. The ophthalmic composition of claim 1, wherein the adenosine receptor antagonist is a non-selective adenosine antagonist.

14. The ophthalmic composition of claim 1, wherein the adenosine receptor antagonist is a xanthine compound is or comprises caffeine, 7-methylxanthine; 1,7-dimethylxanthine (paraxanthine), 3,7-dimethylxanthine (theobromine); 7-methylxanthine (heteroxanthine), 3-methylxanthine; 1-methylxanthine, isobutylmethylxanthine (IBMX); 1-Hexyl-3,7-dimethylxanthine (pentifylline); or 1,7-dimethylxanthine; or a combination thereof.

15. The ophthalmic composition of claim 1, wherein the adenosine receptor antagonist is caffeine or 7-methylxanthine, or a pharmaceutically acceptable salt thereof, such as for example, caffeine citrate.

16. The ophthalmic composition of claim 1, wherein the parasympathetic agent is a cholinergic agent or a nicotinic agent.

17. The ophthalmic composition of claim 1, wherein the parasympathomimetic agent is any combination of one or more of neostigmine, cevimeline, pilocarpine, aceclidine, carbachol, methacholine, echothiophate, physostigmine, prostigmine, phospholine iodide, demecarium bromide and related compounds.

18. The ophthalmic composition of claim 1, wherein the ophthalmic composition is delivered as a solution, suspension, ointment, cream, gel, spray, an extended release system such as those utilizing liposomes, niosomes, nanoparticles, encapsulation, a polymeric matrix systems such as hydrogels, contact lenses, biodegradable materials, molecular printing, an ocular insert or a combination of one or more of the above.

19. The ophthalmic composition of claim 1, wherein the ophthalmic composition is delivered via an extended delivery system.

20. The ophthalmic composition of claim 1, wherein the ophthalmic composition further comprises a preservative and/or a buffer.

21-25. (canceled)
